(19) United States
(12) Patent Application Publication
(10) Pub. No.: US 2026/0284380 A1
(43) Pub. Date: Sep. 24, 2026

(54) ENDOVASCULAR THERAPY DEVICE

(71) Applicant: Covidien LP, Mansfield, MA (US)

(72) Inventors: Haitao Zhang, Maple Grove, MN (US); Yao-Chuan Chang, New Brighton, MN (US); Bernard Q. Li, Plymouth, MN (US); Varun Umesh Kashyap, Palo Alto, CA (US)

(21) Appl. No.: 19/085,603

(22) Filed: Mar. 20, 2025

Related U.S. Application Data
(60) Provisional application No. 63/565,800, filed on Mar. 15, 2024.

(51) Int. Cl. A61N 1/05; A61N 1/36; A61N 1/375

(52) U.S. Cl. CPC A61N 1/05; A61N 1/3605; A61N 1/3754

(57) ABSTRACT

In some examples, a medical lead includes a first lead body portion including a first group of coiled wire filars, a second lead body portion including a second group of coiled wire filars, and third lead body portion including a third group of coiled wire filars, and a plurality of electrodes. In some examples, each electrode is electrically connected to at least one coiled wire filar of the first group of coiled wire filars. In some examples, the first group of coiled wire filars splits to form the second group of coiled wire filars and the third group of coiled wire filars. In some examples, the second group of coiled wire filars is configured to electrically connect to a first feedthrough of a medical device and the third group of coiled wire filars is configured to electrically connect to a second feedthrough of the medical device.

### CROSS-REFERENCE TO RELATED APPLICATIONS

This application claims the benefit of and priority to U.S. Provisional Patent Application Ser. No. 63/565,800 filed Mar. 15, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes and/or senses one or more patient parameters with the aid of the one or more electrodes.

### SUMMARY

This disclosure describes example endovascular medical devices and systems configured to endovascularly deliver electrical stimulation therapy to a patient (e.g., to one or more nerves or brain targets) and/or sense one or more patient parameters (e.g., brain signals and/or other physiological parameters), and related methods. The medical devices and systems described herein are configured to be relatively minimally invasive at least because they are configured to be navigated through vasculature of a patient to a cranial blood vessel (e.g., a venous vessel or an arterial vessel) in order to deliver electrical stimulation therapy to a target tissue site of a patient.

In the examples described herein, an endovascular device (e.g., a medical lead) is configured to a have relatively low-profile yet mechanically robust portion for placement in vasculature of a patient while also having a portion configured to connect a multi-channel medical device outside of the vasculature. The endovascular device includes an elongated lead body including coiled wire filars configured to electrically connect electrodes to a medical device. The lead body includes a first lead body portion including a first group of coiled wire filars. The electrodes are electrically connected to the wire filars of the first group of coiled wire filars. The first group of coiled wire filars splits to form a second group of coiled wire filars and a third group of coiled wire filars. The lead body also includes a second lead body portion including the second group of coiled wire filars and a third lead body portion including the third group of coiled wire filars. The second group of coiled wire filars is configured to electrically connect to a first feedthrough of the medical device and the third group of coiled wire filars is configured to electrically connect to a second feedthrough of the medical device.

Splitting the first group of coiled wire filars into separate groups of coiled wire filars (e.g., the second and third groups of coiled wire filars) enables connection to the medical device via at least two separate feedthroughs. Additionally, maintaining a non-split group of coiled wire filars (e.g., the first group of coiled wire filars) at a distal portion of the lead body (e.g., the first lead body portion) enables the distal portion to maintain a relatively low-profile and/or mechanically robust configuration for placement into the vasculature of the patient. In some examples, the distal portion of the lead body (e.g., the first lead body portion) includes a single multi-filar wire coil and/or a dual concentric multi-filar wire coils, which may be relatively low-profile. The low-profile distal portion of the lead body may facilitate navigation through vasculature (e.g., tortuous cranial vasculature) to relatively distal sites (e.g., in the brain). Additionally, once placed in the vascular sites, the relatively low-profile distal portion of the lead body may reduce occurrence of thrombus as compared to larger (e.g., larger profile) leads or multiple leads. A single multi-filar wire coil and/or a dual concentric multi-filar wire coils configure the distal lead body portion to be sufficiently mechanically robust to withstand relatively high mechanical stress (e.g., as a result of being placed in the vasculature during a medical procedure, or due to movement in a patient after being placed).

In some examples, a medical lead includes a first lead body portion including a first group of coiled wire filars, a second lead body portion including a second group of coiled wire filars, a third lead body portion including a third group of coiled wire filars, and a plurality of electrodes. In some examples, each electrode of the plurality of electrodes is electrically connected to at least one coiled wire filar of the first group of coiled wire filars. In some examples, the first group of coiled wire filars splits to form the second group of coiled wire filars and the third group of coiled wire filars. In some examples, the second group of coiled wire filars is configured to electrically connect to a first feedthrough of a medical device. In some examples, the third group of coiled wire filars is configured to electrically connect to a second feedthrough of the medical device.

In some examples, a method includes introducing a medical lead into vasculature of a patient. In some examples, the medical lead includes a first lead body portion including a first group of coiled wire filars, a second lead body portion including a second group of coiled wire filars, a third lead body portion including a third group of coiled wire filars, and a plurality of electrodes. In some examples, each electrode of the plurality of electrodes is electrically connected to at least one coiled wire filar of the first group of coiled wire filars. In some examples, the first group of coiled wire filars splits to form the second group of coiled wire filars and the third group of coiled wire filars. In some examples, the second group of coiled wire filars is configured to electrically connect to a first feedthrough of a medical device. In some examples, the third group of coiled wire filars is configured to electrically connect to a second feedthrough of the medical device. In some examples, the method includes advancing the medical lead until the plurality of electrodes are adjacent a target location in the vasculature of the patient.

In some examples, a medical device system includes a medical lead including a lead body. In some examples, the medical lead includes a first lead body portion including a first group of coiled wire filars, a second lead body portion including a second group of coiled wire filars, a third lead body portion including a third group of coiled wire filars, a first group of electrical contacts at a proximal portion of the second lead body portion, each electrical contact of the first group of electrical contacts electrically connected to respective filars of the second group of coiled wire filars, a second group of electrical contacts at a proximal portion of the third lead body portion, each electrical contact of the second group of electrical contacts electrically connected to respective filars of the third group of coiled wire filars, a plurality of electrodes, each electrode of the plurality of electrodes electrically connected to at least one coiled wire filar of the first group of coiled wire filars, and an expandable structure at a distal portion of the lead body configured to transform from a relatively low-profile delivery configuration to a deployed configuration in a blood vessel of a patient. In some examples, the plurality of electrodes are disposed on the expandable structure. In some examples, the first group of coiled wire filars splits to form the second group of coiled wire filars and the third group of coiled wire filars. In some examples, the second group of coiled wire filars is configured to electrically connect to a first feedthrough of a medical device via the first group of electrical contacts. In some examples, the third group of coiled wire filars is configured to electrically connect to a second feedthrough of the medical device via the second group of electrical contacts. In some examples, the medical device system includes a medical device configured to control therapy delivery via the plurality of electrodes or sense a patient parameter via the plurality of electrodes.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example therapy system including an endovascular device configured to deliver electrical stimulation therapy to a target tissue site of a patient and/or sense a patient parameter from an endovascular location.

FIG. 2 is a functional block diagram illustrating components of an example medical device of the therapy system of FIG. 1.

FIG. 3 illustrates portions of the example endovascular therapy system of FIG. 1 including an endovascular device that includes a first group of coiled wire filars that splits to form a second group of coiled wire filars and a third group of coiled wire filars.

FIG. 4 illustrates a portion of an example endovascular device of the therapy system of FIG. 1 including a connector configured to connect a first lead body portion to a second lead body portion and a third lead body portion.

FIG. 5 illustrates a portion of an example endovascular device of the therapy system of FIG. 1 including a connector configured to electrically connect a first lead body portion to a second lead body portion and a third lead body portion.

FIG. 6 illustrates an example technique for introducing and advancing an endovascular device according to this disclosure.

Like reference characters denote like elements throughout the description and figures.

### DETAILED DESCRIPTION

The disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson's disease, dystonia, or cerebellar outflow tremor.

While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

In the examples described herein, an endovascular device includes an elongated lead including coiled wire filars configured to electrically connect electrodes to a medical device. The lead includes a first lead body portion including a first group of coiled wire filars that splits to form a second group of coiled wire filars and a third group of coiled wire filars, which facilitates electrical connection of the electrodes to the medical device while maintaining a relatively low-profile distal portion suitable for placement in vasculature of a patient.

The electrodes are electrically connected to the wire filars of the first group of coiled wire filars. The lead also includes a second lead body portion including the second group of coiled wire filars and a third lead body portion including the third group of coiled wire filars. The second group of coiled wire filars is configured to electrically connect to a first feedthrough of a medical device and the third group of coiled wire filars is configured to electrically connect to a second feedthrough of the medical device.

Splitting the first group of coiled wire filars into separate groups of coiled wire filars (e.g., the second group of coiled wire filars and the third group of coiled wire filars) enables connection to the medical device via at least two separate feedthroughs. Splitting the first group of coiled wire filars into separate groups of coiled wire filars can thus facilitate electrical connection to existing medical devices that are configured to receive more than one lead body portion (e.g., via multiple feedthroughs in a header of these existing medical devices). These existing medical devices that are configured to receive multiple lead body portions (e.g., two lead body portions with eight filars each, otherwise referred to as 2×8) may have a relatively small footprint compared to medical devices with a single feedthrough with the same number of filars (e.g., a medical device configured to receive one lead body with sixteen filars, otherwise referred to as 1×16). Additionally, maintaining a non-split group of coiled wire filars (e.g., the first group of coiled wire filars) at a distal portion of the lead body (e.g., the first lead body portion) enables a relatively low-profile (e.g., relatively small diameter or other cross-sectional dimension) and/or mechanically robust portion of the lead for placement into the vasculature of the patient.

In some examples, the distal portion of the lead body (e.g., the first lead body portion) includes a single multi-filar wire coil and/or a dual concentric multi-filar wire coils, which may be relatively low-profile (e.g., low-profile relative to two or more separate, noncoaxial multi-filar wire coils). The low-profile distal portion of the lead body may be deliverable to vascular sites (e.g., via conventional vascular delivery catheters). Additionally, once placed in the vascular sites, the low-profile distal portion of the lead body may reduce occurrence of thrombus as compared to larger (e.g., larger profile) leads or multiple leads. Additionally, the single multi-filar wire coil and/or a dual concentric multi-filar wire coils is sufficiently mechanically robust to withstand relatively high mechanical stress (e.g., as a result of being placed in the vasculature during a medical procedure, or due to movement in a patient after being placed).

In some examples, a medical device is configured to generate electrical stimulation and/or sense a patient parameter via the electrodes of the endovascular device. The electrodes may be carried by or otherwise disposed on an expandable structure, which may be configured to orient the electrodes and/or anchor the electrodes at a particular location in the vasculature of the patient.

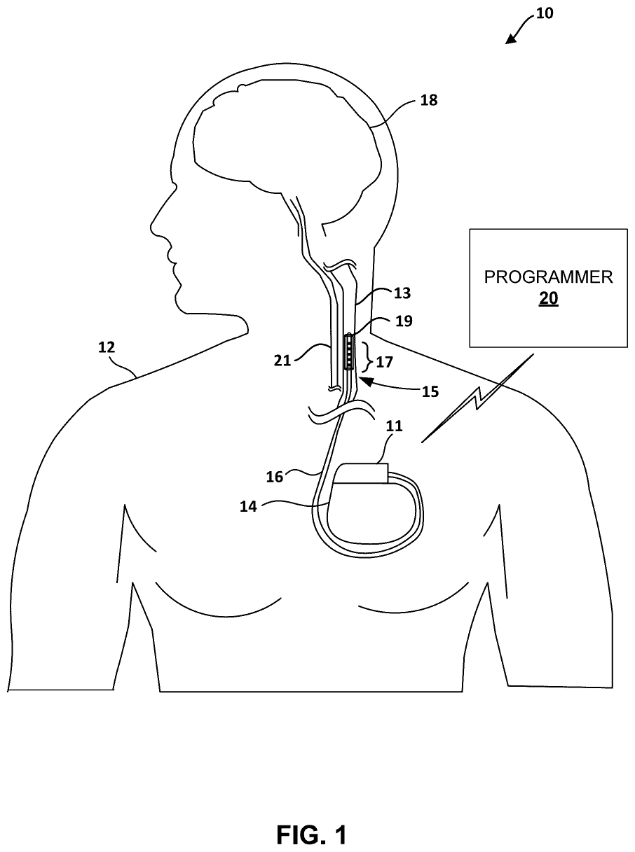

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example shown in FIG. 1, medical device 14 is configured to deliver electrical stimulation therapy (e.g., VNS) to a vagus nerve 21 of patient 12 and/or sense bioelectric signals via electrodes 17. However, in other examples, therapy system 10 and/or medical device 14 is configured to deliver electrical stimulation therapy (e.g., DBS) to brain 18 of patient 12 and/or sense bioelectrical brain signals in brain 18 via electrodes 17. Endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrodes 17 in apposition with a vessel wall (e.g., of jugular vein 13). Medical device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrodes 17. For example, endovascular device 16 can be a medical lead, a catheter, a guidewire, or another elongated body carrying electrodes 17 and configured to be electrically coupled to medical device 14 via an electrically conductive pathway that runs between medical device 14 and electrodes 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples where endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used.

Endovascular device 16 may be used to access jugular vein 13, as well as relatively distal locations in a patient, such as the middle cerebral artery (MCA) in a brain of a patient. Endovascular device 16 may include an elongated body that is structurally configured to be relatively flexible, pushable, and relatively kink-and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands) proximate electrodes 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, etc.) with the aid of a guide member. The guide member can include an outer catheter, an inner catheter, a guide extension catheter, a guidewire, or the like or combination thereof.

In some examples, more than one endovascular device 16 is implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16 can be implanted in a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods, such as to determine, evaluate, or confirm an efficacy of stimulation and/or sensing.

The electrical stimulation therapy described herein (e.g., VNS) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, and obesity. Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, electrodes 17 are positioned on (e.g., coupled to, defined by, or otherwise carried by) expandable structure 19 of endovascular device 16, which is configured to expand radially outwards from a relatively low-profile (e.g., radially compressed) delivery configuration to a deployed configuration. This may enable electrodes 17 to be held in apposition with a blood vessel wall, promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example shown in FIG. 1, endovascular device 16 is directly or indirectly mechanically and electrically coupled to medical device 14 via a header 11 of medical device 14, which defines a plurality of electrical contacts in one or more feedthrough portions for electrically coupling electrodes 17 to electrical stimulation generation circuitry and/or sensing circuitry within medical device 14. As discussed in other portions, header 11 may include multiple feedthrough portions, which may be respectively configured for receiving one of multiple portions of endovascular device 16. Header 11 may also be referred to as a connector block or connector of medical device 14. In the example of FIG. 1, endovascular device 16 is directly coupled to header 11 without the aid of a lead extension. However, in some examples, a lead extension is used between header 11 and endovascular device 16.

In some examples, medical device 14 is configured to be implanted in patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As shown in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include an electrode combination, a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more implantable endovascular devices 16 coupled to medical device 14. The electrode combination may also refer to the polarities of the electrodes in the selected subset. By selecting particular electrode combinations, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

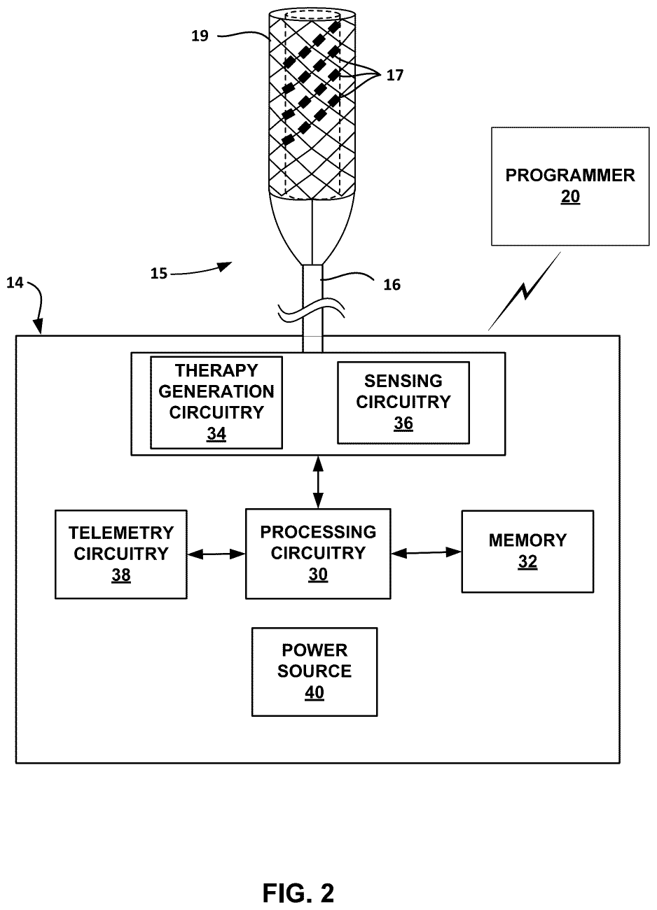

In some examples, in addition to or instead of delivering electrical stimulation to a target location (e.g., vagus nerve 21), medical device 14 or another device senses one or more patient parameters, such as bioelectrical signals, either using electrodes 17 or other types of sensors that are carried by endovascular device 16. Bioelectric signals can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19. In some examples, one or more sensors that are different from electrodes 17 are positioned on the same expandable structure (e.g., expandable structure 19) as one or more electrodes 17 or on a different expandable structure (e.g., expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. Positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables the expandable structure to assume a predetermined shape in the absence of a force (e.g., a compressive or tensile force) holding expandable structure 19 in a relatively low-profile delivery configuration. For example, expandable structure 19 can be configured to expand radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal withdrawal of a pull member attached to a distal portion of the endovascular device 16 or in response to a distal movement of an elongated control member attached to the expandable structure.

Expandable structure 19 can have any suitable configuration in its deployed (e.g., expanded) configuration. For example, expandable structure 19 can be a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or coupled together) or can be a unitary structure (e.g., a laser cut nitinol tube).

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 (as well as electrodes 17, medical device 14, processing circuitry, etc.) may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration than a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more endovascular devices may be used for multiple trial periods (e.g., successive trial periods) for determining an efficacy of one or more stimulation parameters and/or one or more sensing parameters.

Endovascular device 16 may have any suitable configuration for delivering electrical stimulation to a target tissue site in patient 12 or sensing a patient parameter from an endovascular location (e.g., jugular vein 13). For example, endovascular device 16 can include a first body portion (also referred to herein as a first lead body portion) that splits into a second body portion (also referred to herein as a second lead body portion) and a third body portion (also referred to herein as a third lead body portion). The first lead body portion can include a single multi-filar wire coil and/or a dual concentric multi-filar wire coils. The second lead body portion and the third lead body portion can each include single multi-filar wire coils that are not concentric with each other. Said another way, the second lead body portion and the third lead body portion are nonconcentric in some examples. As medical device 14 can include multiple (e.g., two or more) feedthrough portions in header 11, the splitting of the first lead body portion into at least a second lead body portion and the third lead body portion facilitates connection of each of the second lead body portion and the third lead body portion to the feedthrough portion in header 11 of medical device 14. The single elongated body of first body portion of endovascular device 16, which may be a distal portion of endovascular device 16, enables a relatively low-profile (e.g., low-profile as compared to two or more noncoaxial elongated bodies, which may be split along their entire respective lengths) yet mechanically robust portion of endovascular device 16 for positioning within vasculature of patient 12.

While this disclosure primarily describes splitting of medical leads into two lead body portions for connection to a two-feedthrough medical device, it should be understood that the techniques of this disclosure can be extrapolated to splitting any number of leads into any number of separate lead portions. For example, a single medical lead (e.g., endovascular device 16) may be split into two, three, four, fix, six, or more portions, including respective groups of wire filars, for connection one or more medical devices.

In some examples, therapy system 10 includes a connector (not shown in the example of FIG. 1) configured to electrically connect the first lead body portion to the second lead body portion and the third lead body portion. The connector portion may be placed in a suitable location, such as outside the vasculature. The connector may be configured to electrically connect a portion of the first lead body portion to portions of the second lead body portion and the third lead body portion. Although in some examples herein, the term “split” is used herein to describe the individual wire filars of the first lead body portion being connected to individual wire filars of separate coils of the second lead body portion and the third lead body portion, even in some of the “split” examples, the connectors described in this disclosure can be configured to electrically connect some individual wire filars of the first lead body portion to the individual wire filars of the second lead body portion, as well as connect other individual wire filars of the first lead body portion to the individual wire filars of the third lead body portion, thus “splitting” the first lead body portion into a second lead body portion and a third lead body portion. Thus, when a first group of wire filars is split into second and third groups of wire filars, wire filars of the first and second groups can be continuous (e.g., unbroken) wire filars or the wire filars of the first group may be physically separate from the wire filars of the second group and electrically connected to the wire filars of the second group via a connector. Similarly, the wire filars of the first and third groups can be continuous wire filars or the wire filars of the first group may be physically separate from the wire filars of the third group and electrically connected to the wire filars of the third group via a connector.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectric signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 27 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

As discussed elsewhere, in some examples, endovascular device 16 is configured to be a standalone electrical stimulation device and can include one or more elements of medical device 14 shown in FIG. 2.

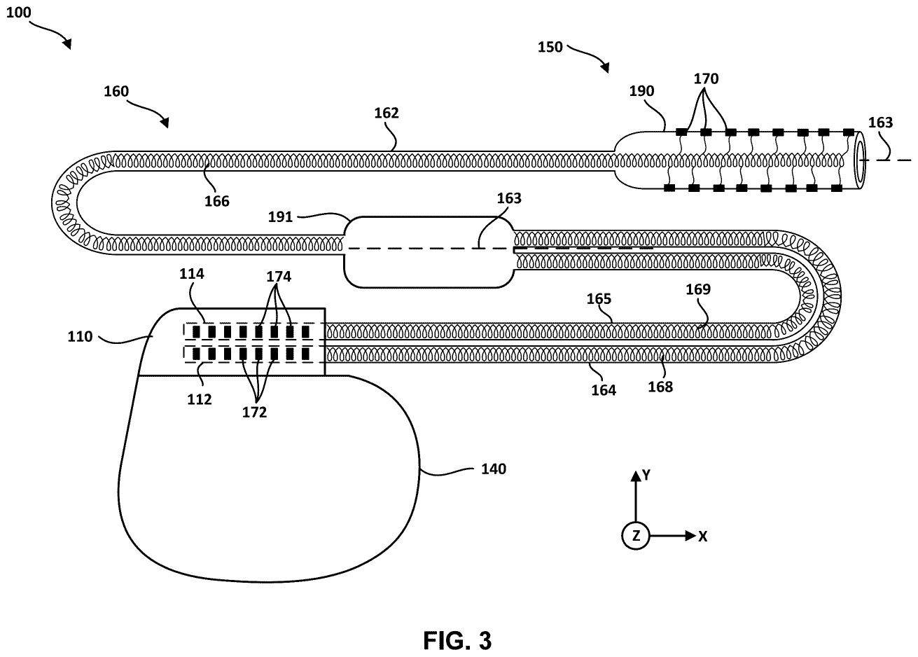

FIG. 3 illustrates an example endovascular therapy system 100, which may be an example of therapy system 10 of FIG. 1. Endovascular therapy system 100 includes a medical device 140, a medical lead 160, a plurality of electrodes 170 (which may be examples of medical device 14, endovascular device 16, and electrodes 17, respectively, as shown and described in connection with FIG. 1, respectively). In the example of FIG. 3, medical lead 160 includes a first lead body portion 162, which may be a distal portion (e.g., a distal-most portion) of lead 160. Additionally, medical lead 160 includes and a second lead body portion 164 and a third lead body portion 165, which together may be a proximal portion (e.g., a proximal-most portion) of lead 160.

First lead body portion 162 includes a first group of coiled wire filars 166, second lead body portion 164 includes a second group of coiled wire filars 168, and third lead body portion 165 includes a third group of coiled wire filars 169. In some examples, as illustrated, first group of coiled wire filars 166 splits to form second group of coiled wire filars 168 and third group of coiled wire filars 169. This splitting may facilitate connection of the wire filars to medical device 140 via a first feedthrough 112 and a second feedthrough 114 of header 110 of medical device 140 while maintaining a single multi-filar coil or dual concentric coil configuration in the first lead body portion 162 (e.g., which may be a distal portion of lead 160). The single multi-filar coil or dual concentric coil configuration in the first lead body portion 162 (e.g., which may be a distal portion) may enable a relatively low-profile (e.g., low profile compared to two or more noncoaxial coils or body portions, which may be split along their entire respective lengths) yet mechanically robust portion of medical lead 160 that is configured to reside and even remain implanted in the vasculature for chronic therapy delivery. In some examples, some or all of the second lead body portion 164 and/or third lead body portion 165 are configured to be positioned outside of the vasculature an connect to medical device 140, which may be placed in an extravascular portion of the chest or external to patient 12.

In the example of FIG. 3, second group of coiled wire filars 168 is configured to electrically connect to a first feedthrough 112 of header 110 of a medical device 140, and third group of coiled wire filars 169 is configured to electrically connect to a second feedthrough 114 of header 110 of a medical device 140. In some examples, lead 160 includes a first group of electrical contacts 172 are electrically connected to filars of second group of coiled wire filars 168 and facilitate electrical connection of at least some electrodes 170 to medical device 140 via first feedthrough 112. In some examples, each electrical contact of first group of electrical contacts 172 is electrically connected to a respective filar of second group of coiled wire filars 168. Similarly, in some examples, lead 160 includes a second group of electrical contacts 174 are electrically connected to filars of third group of coiled wire filars 169 and facilitate electrical connection of at least some electrodes 170 to medical device 140 via second feedthrough 114. In some examples, each electrical contact of second group of electrical contacts 174 is electrically connected to a respective filar of third group of coiled wire filars 169.

In some examples, first group of electrical contact 172 and second group of electrical contacts 174 are configured to engage respective features of first feedthrough 112 and second feedthrough 114, such as for electrically connecting filars of second group of wire filars 168 and third group of wire filars 169 to circuitry (e.g., processing circuitry, therapy generation circuitry, sensing circuitry, as discussed in relation to FIG. 2) of medical device 140. In this way, second group of coiled wire filars 168 is configured to electrically connect to first feedthrough 112 of medical device 140 via first group of electrical contacts 172 and third group of coiled wire filars 169 is configured to electrically connect to second feedthrough 114 of the medical device via second group of electrical contacts 174. In some examples, first group of electrical contacts 172 are disposed on a proximal portion of second lead body portion 164. In some examples, second group of electrical contacts 174 are disposed on a proximal portion of third lead body portion 165.

In some examples, second lead body portion 164 and/or third lead body portion 165 are configured to mechanically couple to header 110. In some examples, header 110 may be configured to receive portions of second lead body portion 164 and/or third lead body portion 165 and/or retain portions of second lead body portion 164 and/or third lead body portion 165 within header 110. In some examples, second lead body portion 164, third lead body portion 165 and/or header 110 include features (e.g., mating features) to facilitate mechanical coupling (e.g., snap fit features). In some examples, header 110 includes one or set screws for mechanically coupling second lead body portion 164 and/or third lead body portion 165 to header 110. Second lead body portion 164, third lead body portion 165 and/or header 110 can include any suitable features for mechanical connection, including Bal Seal® connectors, spring connectors, push fittings, or other suitable connectors.

As shown in the example of FIG. 3, second group of coiled wire filars 168 and third group of coiled wire filars 169 are not coaxial (e.g., nonconcentric), which may enable connection to separate feedthroughs of medical device 140 (e.g., first feedthrough 112 and second feedthrough 114, which may be separate feedthroughs). In some examples, second group of coiled wire filars 168 and third group of coiled wire filars 169 are noncoaxial along the entire length of each of second group of coiled wire filars 168 and third group of coiled wire filars 169. In other examples, some or all of second group of coiled wire filars 168 and third group of coiled wire filars 169 may be wound around each other (e.g., in a double helix configuration). In some examples, a longitudinal axis of each of second group of coiled wire filars 168 and third group of coiled wire filars 169 are parallel, such as when second lead body portion 164 and third lead body portion 165 extent straight away from header 110 (e.g., straight away from first feedthrough 112 and second feedthrough 114 of header 110, respectively).

Although second body portion 164 and third lead body portion 165, which contain second group of coiled wire filars 168 and third group of coiled wire filars 169 respectively, are shown as physically separate elongated bodies in the example of FIG. 3, in some examples, some or all of second group of coiled wire filars 168 and third group of coiled wire filars 169 may be contained in a single elongated body. Additionally, although the example of FIG. 3 illustrates second lead body portion 164 and third lead body portion 165, as extending some distance between connector 191 and header 110 of medical device 140, these portions may be entirely contained within one or more of connector 191 and/or header 110. In other words, in some examples, connector 191 is configured to interface directly with header 110 (including first feedthrough 112 and/or second feedthrough 114).

In some examples, some or all of wire filars of first group of coiled wire filars 166, second group of coiled wire filars 168, and/or third group of coiled wire filars 169 include a material or combination of materials configured to facilitate relatively high flexibility, high axial extensibility, and/or high fatigue resistance. For examples, one or more filars of first group of coiled wire filars 166, second group of coiled wire filars 168, and/or third group of coiled wire filars 169 includes a beta-titanium alloy. In some examples, the beta-titanium alloy comprises a Ti-15Mo alloy. Certain beta-titanium alloys, including Ti-15Mo alloy and similar titanium alloys enable higher count filar coils (e.g., twelve filar or greater, including sixteen filar), such as for situations where a relatively high number of individually controlled electrodes are needed in a small space including nerve stimulation and/or sensing from endovascular locations. In some examples, one or more of the filars includes a core material (e.g., a core at a radial center of each filar). The core material can be configured to enhance mechanical robustness. In some examples, the core material includes tantalum.

In some examples, a proximal portion and/or a distal portion of lead 160, which may include some or all of wire filars of first group of coiled wire 166, second group of coiled wire filars 168, and/or third group of coiled wire filars 169, includes a beta-titanium alloy (e.g., which may be a second, different beta-titanium alloy than the wire filars noted above). For example, respective proximal and/or distal portions of one or more of first lead body portion 162, second lead body portion 164 and/or third lead body portion 165 can include the beta-titanium alloy. The beta-titanium alloy may facilitate welding. In some examples, the beta-titanium alloy includes TiTaSn. In some examples, the beta-titanium alloy portion may function as a connection ring for welding to the wire filars (e.g., in examples where the wire filars include Ti-15Mo).

In some examples, lead 160 includes an electrically insulative material covering at least some portions of lead 160. In some examples, one or more of first lead body portion 162, second lead body portion 164 and/or third lead body portion 165 include an electrically insulative material over at least a portion of the coils formed by the coiled wire filars of each of first lead body portion 162, second lead body portion 164 and/or third lead body portion 165. In some examples, the electrically insulative material includes a tubular-like polymeric covering over one or more portions first lead body portion 162, second lead body portion 164 and/or third lead body portion 165. In some examples, lead 160, including one or more portions of first lead body portion 162, second lead body portion 164 and/or third lead body portion 165 includes polyurethane of a suitable durometer (e.g., 55 D Shore D hardness). Additionally or alternatively, some or all individual wire filars of first group of coiled wire filars 166, second group of coiled wire filars 168, and/or third group of coiled wire filars 169 include electrically insulative coatings and/or electrically insulative materials disposed over portions of individual wire filars. In some examples, some or all individual wire filars of first group of coiled wire filars 166, second group of coiled wire filars 168, and/or third group of coiled wire filars 169 include Si-polyamide coating.

As described herein, each of first group of coiled wire filars 166, second group of coiled wire filars 168, and/or third group of coiled wire filars 169 include coils that form tubular, wire-like structure. For example, each group of coiled wire filars may generally form an elongated tube extending along a longitudinal axis and a defining a maximum dimension extending away from the longitudinal axis. In some examples, the coils define a pitch (e.g., an angle of each individual wire filar relative to a longitudinal axis of the coil) and/or an axial spacing between adjacent individual filars. In some examples, an axial spacing between individual adjacent wire filars of a respective coil is less than a maximum cross-sectional dimension (e.g., diameter) of the individual wire filars. Although the examples of this disclosure include groups of wire filars as coils, other configurations are possible, such as, but not limited to, bundles, twisted bundles, parallel wire bundles, or other suitable configurations of groups of individual wire filars.

In some examples, one or more of first group of coiled wire filars 166, second group of coiled wire filars 168, and/or third group of coiled wire filars 169 define coils that enable other devices (e.g., guidewires) to be inserted through a lumen of the coils. For example, a coil includes an inner lumen with a diameter large enough to allow a guidewire (or another device) to be inserted into and pass through the lumen.

Together, first group of coiled wire filars 166, second group of coiled wire filars 168, and/or third group of coiled wire filars 169 facilitate electrical connection of electrodes 170 to medical device 140. In some examples, each of electrodes 170 is configured to be individually (e.g., separately, independently) controlled via medical device 140 to deliver electrical stimulation therapy and/or sense a patient parameter. In the example of FIG. 3, each electrode of electrodes 170 is electrically connected to at least one coiled wire filar of first group of coiled wire filars 166. Because of the relatively high-filar count of first group of coiled wire filars 166, a relatively high number of corresponding electrodes may be connected to and used in conjunction with medical device 140, while still maintaining a relatively low profile (e.g., relative to two separate, noncoaxial groups of coiled wire filars) that facilitates positioning of electrodes 170 in vasculature of a patient. In some examples, electrodes 170 include sixteen electrodes, although other suitable numbers of electrodes are provided in other examples (e.g., four, six, eight, twelve, fourteen, sixteen, eighteen, twenty, or more electrodes). In some examples, a number of electrodes of electrodes 170 equals a number of wire filars of first group of wire filars 166 (e.g., sixteen electrodes for sixteen wire filars). However, in other examples, more than one electrode of electrodes 170 is connected to each filar, such that a number of electrodes is greater than a number of wire filars of first group of wire filars 166 (e.g., in examples where at least some electrodes are shorted or ganged together).

As discussed in relation to previous examples, lead 160 can include an expandable structure 190 at a distal portion 150 of lead 160 to position and/or orient electrodes 170 within vasculature of a patient. In some examples, electrodes 170 are carried by, disposed on, and/or defined by expandable structure 190 which is configured to transform from a relatively low-profile delivery configuration to a deployed configuration in a blood vessel of a patient (e.g., within jugular vein 13 of patient 12 as discussed in relation to FIG. 1 or in a blood vessel in brain 18 (FIG. 1) of patient 12). Expandable structure 190 may be an example of expandable structure 19 as discussed in connection with FIG. 1, and can include any suitable shape and materials. In some examples, expandable structure 190 includes one or more of a self-expanding stent, a balloon, a self-expanding coil, or another suitable expandable structure as described herein.

In some examples, lead 160 includes a connector 191 configured to connect first group of coiled wire filars 166 of first lead body portion 162 to second group of coiled wire filars 168 and third group of coiled wire filars 169 of second lead body portion 164 and third lead body portion 165, respectively. For example, as discussed herein, connector 191 can include components for electrically coupling individual wire filars of first group of coiled wire filars 166 to individual wire filars of second group of coiled wire filars 168 and/or third group of coiled wire filars 169. In this way, connector 191 is configured to facilitate “splitting” of first group of coiled wire filars 166 into second group of coiled wire filars 168 and/or third group of coiled wire filars 169. Said another way, connector 191 is configured to “join” second group of coiled wire filars 168 and third group of coiled wire filars 169 into first group of coiled wire filars 166.

In the example of FIG. 3, a proximal portion of first lead body portion 162 is mechanically coupled to a distal portion of connector 191, and respective distal portions of second lead body portion 164 and third lead body portions 165 are mechanically coupled to a proximal portion of connector 191. In some examples, connector 191 is configured to received portions of first lead body portion 162, second lead body portion 164, and third lead body portions 165 (e.g., via holes, ports, feedthroughs, or the like). However, in other examples, connector 191 is integrally formed as part of first lead body portion 162, second lead body portion 164, and/or third lead body portion 165. Further, connector 191 may additionally or alternatively be integrally formed as part of expandable structure 190, header 110, and/or medical device 140.

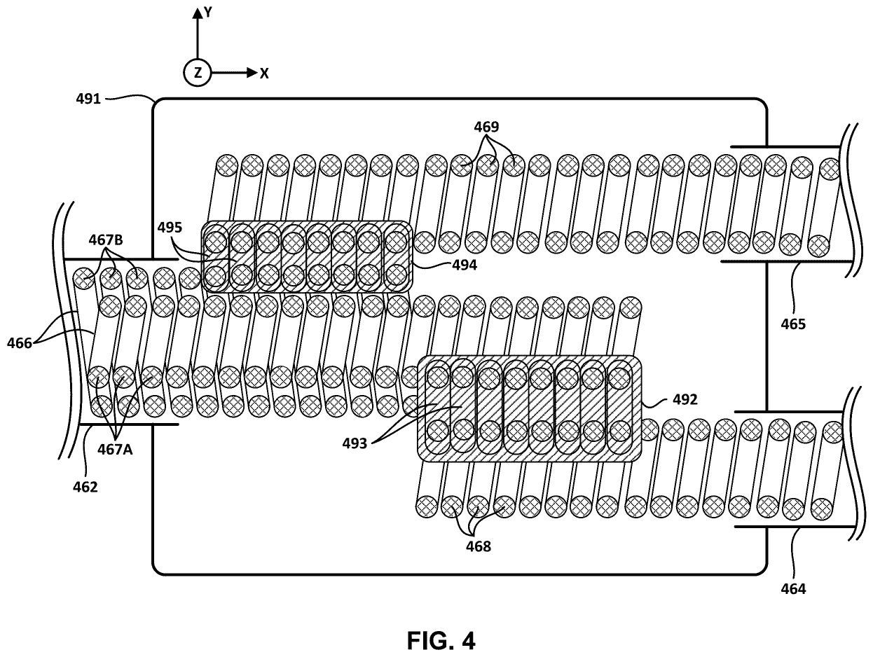

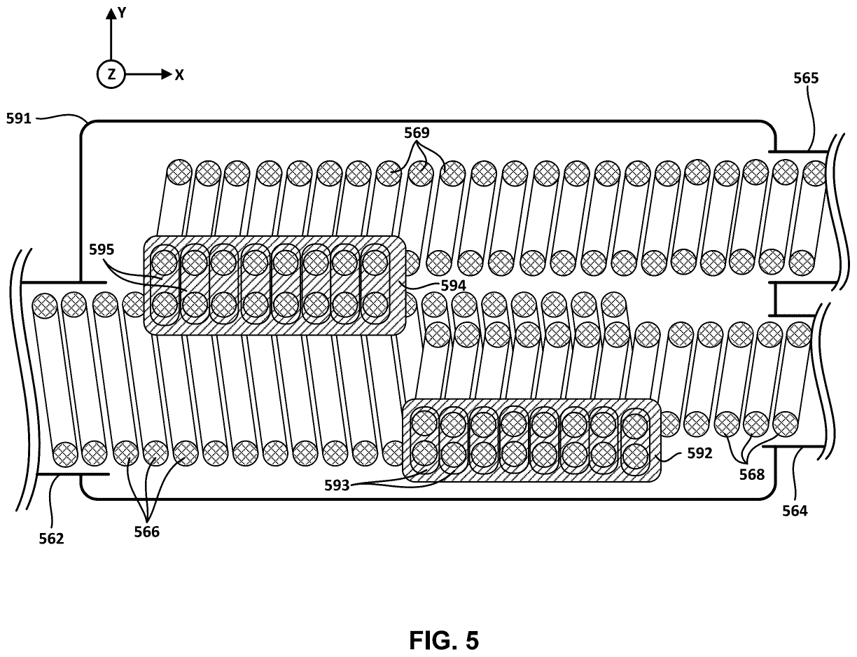

FIG. 4 and FIG. 5 illustrate example connector 491 and example connector 591 respectively, which may be examples of connector 191 as illustrated and described in connection with FIG. 3. In particular, FIG. 4 and FIG. 5 illustrate cross-sections of connectors, as well as groups of filars that form coils, the cross-section taken through a plane parallel to a longitudinal axis 163 of the device (e.g., through a x-y plane, where orthogonal x-y-z axes are shown in the figures for ease of description).

In the example of FIG. 4, connector 491 is configured to electrically connect a first group of coiled wire filars 466 (which may be an example of first group of coiled wire filars 166 in the example of FIG. 3) to a second group of coiled wire filars 468 and a third group of coiled wire filars 469 (which may be examples of second group of wire filars 168 and third group of wire filars 169, respectively, in the example of FIG. 3). Connector 491 facilitates splitting of first group of coiled wire filars 466 of a first lead body portion 462 to form second group of coiled wire filars 468 of a second lead body portion 464 and third group of coiled wire filars 469 of a third lead body portion 465.

While the example of FIG. 4 illustrates splitting first lead body portion 462 into two separate lead body portions (second lead body portion 464 and third lead body portion 465), the techniques of this disclosure may enable splitting of first lead body portion 462 into any suitable number of separate lead body portions. In some examples, first lead body portion 462 (and first group of coiled wire filars 466) splits to form more than two separate lead body portions and corresponding groups of coiled wire filars. In some examples, first lead body portion 462 (and first group of coiled wire filars 466) splits to form three, four, five, six, seven, eight, nine, ten, twelve, or more lead body portions and corresponding groups of coiled wire filars.

In the example of FIG. 4, first group of coiled wire filars 466 comprises dual concentric coils including an inner coil 467A and an outer coil 467B. The dual coil configuration may enhance flexibility, axial extensibility, and/or fatigue resistance of first lead body portion 462 while maintaining a sufficiently small form-factor (e.g., a relatively small cross-sectional area of the dual coils as a whole). In some examples, as shown in the example of FIG. 4, inner coil 467A and outer coil 467B are wound in opposite directions (e.g., different directions relative to each other, such as clockwise & counterclockwise). Winding the wire filars such that inner coil 467A and an outer coil 467B are wound in opposite directions may permit movement of the inner coil 467A relative to outer coil 467B, and may permit the first group of coiled wire filars 466 to be bent with a relatively small radius of curvature without permanent or lasting deformation of first lead body portion 462.

The dual coil configuration of inner coil 467A and an outer coil 467B also permit the first group of coiled wire filars 466 to elastically expand in a longitudinal direction (e.g., along a central longitudinal axis of inner coil 467A and outer coil 467B, such as parallel to the x-axis direction in the example of FIG. 4). The dual coil configuration of inner coil 467A and an outer coil 467B wound in different directions may also enhance torque transfer (e.g., in at least lead body portion 462). The increased torque transfer may facilitate navigation of the lead through vasculature during implantation (e.g., of at least lead body portion 462). However, in other examples the dual concentric coils of inner coil 467A and outer coil 467B are wound in a common directions (e.g., the same direction relative to each other).

As with other examples described herein, the dual coil configuration of first group of coiled wire filars 466 may facilitate a relatively high number of individual filars in each coil while maintaining a relatively small form factor (e.g., low-profile) of first group of coiled wire filars 466 and/or first lead body portion 462. In some examples, each of inner coil 467A and outer coil 467B includes at least six filars. In some examples, each of inner coil 467A and outer coil 467B includes at least eight filars. However, in other examples, each of inner coil 467A and outer coil 467B can include any suitable number of filars (e.g., four, six, eight, ten, twelve, fourteen, sixteen, or more). In some examples, each of inner coil 467A and outer coil 467B includes the same number of filars. In some examples, each of inner coil 467A and outer coil 467B includes a different number of filars. In some examples, inner coil 467A includes more filars than outer coil 467B. In some examples, outer coil 467B includes more filars than inner coil 467A. In some examples, each of inner coil 467A and outer coil 467B includes an even number of filars. In some examples, each of inner coil 467A and outer coil 467B includes an odd number of filars.

In some examples, connector 491 includes a plurality of individual connector elements configured to electrically connect individual wire filars of first group of coiled wire filars 466 to respective individual wire filars of the second group of coiled wire filars 468 and third group of coiled wire filars 469. In the example of FIG. 4, connector 491 includes a first connector element 492 configured to electrically connect a first portion of first group of coiled wire filars 466 to second group of coiled wire filars 468. In particular, in the example shown in FIG. 4, first connector element 492 is configured to electrically connect filars of inner coil 467A to second group of coiled wire filars 468. Connector 491 also includes a second connector element 494 configured to electrically connect a second portion of first group of coiled wire filars 466 to third group of coiled wire filars 469. In particular, in the example shown in FIG. 4, second connector element 494 is configured to electrically connect filars of outer coil 467B to third group of coiled wire filars 469. In this way, first connector element 492 and second connector element 494 are configured to electrically connect filars of first group of coiled wire filars 466 to filars of second group of coiled wire filars 468 and third group of coiled wire filars 469.

In some examples, each of first connector element 492 and/or second connector element 494 include electrically conductive portions configured to contact individual filars of each of first group of coiled wire filars 466, second group of coiled wire filars 468, and/or third group of coiled wire filars 469. In some examples, each of first group of coiled wire filars 466, second group of coiled wire filars 468, and/or third group of coiled wire filars 469 include configurations suitable for electrically connecting individual filars (e.g., via contact elements, which may include exposed portions of individual filar wires, etc.) to other elements (e.g., first connector element 492 and/or second connector element 494). In some examples, first lead body portion 462 includes a set of contact elements (e.g., at a proximal portion of first lead body portion 462) that are connected to respective filars of first group of coiled wire filars 466 and configured to be received in connector 491. In some examples, the set of contact elements of first lead body portion 462 are configured to be received in and/or contacted by one or more of first connector element 492 and/or second connector element 494.

In some examples, second lead body portion 464 includes a set of contact elements (e.g., at a distal portion of second lead body portion 464) that are connected to respective filars of second group of coiled wire filars 468 and configured to be received in connector 491. In some examples, the set of contact elements of second lead body portion 464 are configured to be received in and/or contacted by first connector element 492.

In some examples, third lead body portion 465 includes a set of contact elements (e.g., at a distal portion of third lead body portion 465) that are connected to respective filars of third group of coiled wire filars 469 and configured to be received in connector 491. In some examples, the set of contact elements of third lead body portion 465 are configured to be received in and/or contacted by second connector element 494.

In some examples, first connector element 492 includes a plurality of individual filar contact portions 493. Each of the individual filar contact portions 493 may be configured to contact and/or electrically connect an individual filar of first group of coiled wire filars 466 and an individual wire filar of second group of coiled wire filars 468. Each individual filar contact portion 493 can be electrically insulated from the other filar contact portions 493. In the example of FIG. 4, where inner coil 467A of first group of coiled wire filars 466 includes eight wire filars (e.g., an octofilar coil), first connector element 492 includes eight individual filar contact portions 493, which also corresponds to eight filars of second group of coiled wire filars 468. In other words, first connector element 492 includes a corresponding number of individual filar contact portions 493 as the number of individual filars in inner coil 467A of first group of coiled wire filars 466 and/or the number of individual filars of second group of coiled wire filars 468.

In some examples, second connector element 494 includes a plurality of individual filar contact portions 495. Each of the individual filar contact portions 495 may be configured to contact and electrically connect an individual filar of first group of coiled wire filars 466 and an individual filar of third group of coiled wire filars 469. Each individual filar contact portion 495 can be electrically insulated from the other filar contact portions 495. In the example of FIG. 4, where outer coil 467B of first group of coiled wire filars 466 includes eight wire filars (e.g., an octofilar coil), second connector element 494 includes eight individual filar contact portions 495, which also corresponds to eight wire filars of third group of coiled wire filars 469. In other words, second connector element 494 includes a corresponding number of individual filar contact portions 495 as the number of individual filars in outer coil 467B of group of coiled wire filars 466 and/or the number of individual filars of third group of coiled wire filars 469.

First connector element 492, second connector element 494, as well as individual filar contact portions 493 and individual filar contact portions 495 can include any suitable components or combination of components configured to electrically connect individual filars of first group of coiled wire filars 466 to second group of coiled wire filars 468 and/or third group of coiled wire filars 469. In some examples, individual filar contact portions 493 and individual filar contact portions 495 include rings comprising electrically conductive materials. Each ring may be configured to contact a first filar from a first group of filars (e.g., from first group of coiled wire filars 466) and a second filar from a second group of filars (second group of coiled wire filars 468 or third group of coiled wire filars 469), such as to electrically connect the first filar from a first group of filars to the second filar from a second group of filars. The rings can be electrically insulated from the adjacent ring(s). Other configurations of conductive materials can be used to engage individual filars, including, but not limited to, Bal Seal® connectors, spring connectors, push fittings, or other suitable connectors. In some examples, first connector element 492 and/or second connector element 494 include groups of rings of conductive material.

While the example of FIG. 4 shows each filar of inner coil 467A connected to second group of coiled wire filars 468 and each filar of outer coil 467B connected to third group of coiled wire filars 469, other arrangements are possible. For example, some of all filars of inner coil 467A can be electrically connected to some or all filars of third group of coiled wire filars 469 in addition to or instead of second group of coiled wire filars 468 and/or some or all filars of outer coil 467B can electrically connected to some or all filars of second group of coiled wire filars 468 in addition to or instead of third group of coiled wire filars 469.

In some examples, the arrangement of inner coil 467A and an outer coil 467B formed by first group of coiled wire filars 466 facilitates connection of first group of coiled wire filars 466 to each of second group of coiled wire filars 468 and third group of coiled wire filars 469. For example, as shown in the example of FIG. 4, inner coil 467A extends proximally of outer coil 467B such that individual wire filars of inner coil 467A are exposed to connect to second group of coiled wire filars 468. In particular, at least a portion of each filar of inner coil 467A extends proximally of a proximal end of outer coil 467B to enable connection to second group of coiled wire filars 468 via first connector element 492. However, inner coil 467A and outer coil 467B of first group of coiled wire filars 466 may have any suitable relative position and/or orientation (e.g., to facilitate connection of first group of coiled wire filars 466 to each of second group of coiled wire filars 468 and third group of coiled wire filars 469).

In the example of FIG. 5, connector 591 is configured to electrically connect a first group of coiled wire filars 566 (which may be an example of first group of coiled wire filars 166 in the example of FIG. 3) to a second group of coiled wire filars 568 and a third group of coiled wire filars 569 (which may be examples of second group of wire filars 168 and third group of wire filars 169, respectively, in the example of FIG. 3). Connector 591 facilitates splitting of first group of coiled wire filars 566 of a first lead body portion 562 to form second group of coiled wire filars 568 of a second lead body portion 564 and third group of coiled wire filars 569 of a third lead body portion 565.

While the example of FIG. 5 illustrates splitting first lead body portion 562 into two separate lead body portions (second lead body portion 564 and third lead body portion 565), the techniques of this disclosure may enable splitting of first lead body portion 562 into any suitable number of separate lead body portions. In some examples, first lead body portion 562 (and first group of coiled wire filars 566) splits to form more than two separate lead body portions and corresponding groups of coiled wire filars. In some examples, first lead body portion 562 (and first group of coiled wire filars 566) splits to form three, four, five, six, seven, eight, nine, ten, twelve, or more lead body portions and corresponding groups of coiled wire filars.

In the example of FIG. 5, first group of coiled wire filars 566 comprises a single coil (e.g., a single multi-filar coil). The single coil form factor of first group of coiled wire filars 566 may be configured to maintain a sufficiently small form-factor (e.g., a relatively cross-sectional area of the coil as a whole) without sacrificing flexibility, axial extensibility, and/or fatigue resistance in first lead body portion 562 (which may be a distal portion, such as described with respect to first lead body portion 162 in FIG. 3)

The single coil configuration of first group of coiled wire filars 566 can facilitate a relatively high number of individual filars in the coil. In some examples, the single coil includes at least twelve filars (e.g., twelve filars). In some examples, the single coil includes at least sixteen filars (e.g., sixteen filars). However, first group of coiled wire filars 566 that includes a single coil configuration can include any suitable number of filars (e.g., four, six, eight, ten, twelve, fourteen, sixteen, twenty, thirty, or more).

In some examples, connector 591 includes a plurality of individual connector elements configured to electrically connect individual filars of first group of coiled wire filars 566 to respective individual filars of the second group of coiled wire filars 568 and third group of coiled wire filars 569. In the example of FIG. 5, connector 591 includes a first connector element 592 configured to electrically connect a first portion of first group of coiled wire filars 566 to second group of coiled wire filars 568. In particular, first connector element 592 is configured to electrically connect a proximal set of first group of coiled wire filars 566 to second group of coiled wire filars 568. Connector 591 also includes a second connector element 594 configured to electrically connect a second portion of first group of coiled wire filars 566 to third group of coiled wire filars 569. In particular, second connector element 594 is configured to electrically connect a distal set of first group of coiled wire filars 566 (e.g., distal to the proximal set of filars described above) to third group of coiled wire filars 569. In this way, first connector element 592 and second connector element 594 are configured to electrically connect filars of first group of coiled wire filars 566 to filars of second group of coiled wire filars 568 and third group of coiled wire filars 569.

In some examples, each of first connector element 592 and/or second connector element 594 include portions configured to contact individual filars of each of first group of coiled wire filars 566, second group of coiled wire filars 568, and/or third group of coiled wire filars 569. In some examples, each of first group of coiled wire filars 566, second group of coiled wire filars 568, and/or third group of coiled wire filars 569 include configurations suitable for electrically connecting individual filars (e.g., via contact elements, which may include exposed portions of individual filar wires, etc.) to other elements (e.g., first connector element 592 and/or second connector element 594). In some examples, first lead body portion 562 includes a set of contact elements (e.g., at a proximal portion of first lead body portion 562) that are connected to respective filars of first group of coiled wire filars 566 and configured to be received in connector 591. In some examples, the set of contact elements of first lead body portion 562 are configured to be received in and/or contacted by one or more of first connector element 592 and/or second connector element 594.

In some examples, second lead body portion 564 includes a set of contact elements (e.g., at a distal portion of second lead body portion 564) that are connected to respective filars of second group of coiled wire filars 568 and configured to be received in connector 591. In some examples, the set of contact elements of second lead body portion 564 are configured to be received in and/or contacted by first connector element 592.

In some examples, third lead body portion 565 includes a set of contact elements (e.g., at a distal portion of third lead body portion 565) that are connected to respective filars of third group of coiled wire filars 569 and configured to be received in connector 591. In some examples, the set of contact elements of third lead body portion 565 are configured to be received in and/or contacted by second connector element 594.

In some examples, first connector element 592 includes a plurality of individual filar contact portions 593. Each of the individual filar contact portions 593 may be configured to contact and/or electrically connect an individual filar of first group of coiled wire filars 566 and an individual filar of second group of coiled wire filars 568. In the example of FIG. 5, where first connector element 592 connects eight filars of first group of coiled wire filars 566 to eight filars of second group of coiled wire filars 568, first connector element 592 includes eight individual filar contact portions 593. In other words, first connector element 592 includes at least a corresponding number of individual filar contact portions 593 as the number of individual filars of second group of coiled wire filars 568. Each individual filar contact portion of the plurality of filar contact portions 593 can be electrically insulated from the other filar contact portions 593.

In some examples, second connector element 594 includes a plurality of individual filar contact portions 595. Each of the individual filar contact portions 595 may be configured to contact and electrically connect an individual filar of first group of coiled wire filars 566 and an individual filar of third group of coiled wire filars 569. In the example of FIG. 5, where second connector element 594 connects eight filars of first group of coiled wire filars 566 to eight filars of third group of coiled wire filars 569, second connector element 594 includes eight individual filar contact portions 595. In other words, second connector element 594 includes at least a corresponding number of individual filar contact portions 595 as the number of individual filars of third group of coiled wire filars 569. Each individual filar contact portion of the plurality of filar contact portions 595 can be electrically insulated from the other filar contact portions 595.

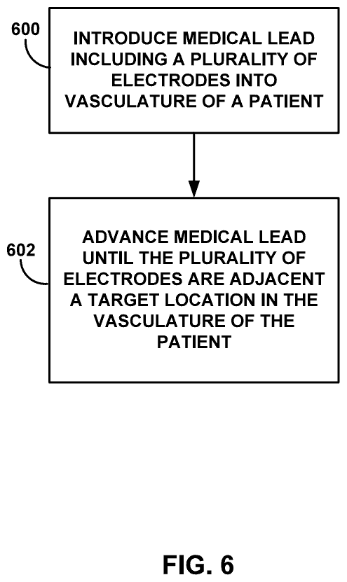

FIG. 6 is a flow diagram illustrating an example technique for using a medical device system according to the techniques of this disclosure, which may include placing a medical lead adjacent a target location in vasculature of a patient. The technique of FIG. 6 is described with respect to therapy system 10 of FIG. 1, as well as endovascular therapy system 100 of FIG. 3 (which is an example of therapy system 10 of FIG. 1), but may be used with any of the device, systems, and/or elements of systems described in this disclosure.

In the example of FIG. 6, the technique includes introducing an endovascular device 16 (which may be a medical lead) into vasculature of patient 12 (600). For example, a clinician introduces at least distal portion 15 of endovascular device 16 through an access point in patient 12 including a femoral artery access point or radial artery access point. In some examples, one or more of an introducer sheath or guide catheter is used to facilitate introduction of endovascular device 16 into patient 12.

In the example of FIG. 6, the technique further includes advancing endovascular device 16 (which may include a medical lead) through the vasculature of the patient until electrodes 17 are adjacent a target location in the vasculature of patient 12 (602). In some examples, a clinician advances endovascular device 16 through vasculature of patient 12 until electrodes 17 are located within jugular vein 13 and positioned adjacent vagus nerve 21. Once electrodes 17 are adjacent the target location (e.g., vagus nerve 21), the clinician initiates (e.g., via programmer 20, or another suitable device) electrical stimulation therapy and/or sensing of one or more patient parameters by medical device 14.

Endovascular device 16 can be placed to position a relatively low-profile portion yet mechanically robust portion in the vasculature of patient 12, while a portion of endovascular device configured to connect to multi-channel header 11 of medical device 14 remains outside of the vasculature of patient 12. In some examples, a distal portion of endovascular device 16 (e.g., first body portion 162, as described in connection with FIG. 3) includes a single low-profile elongated body, and is placed at least partially in the vasculature of patient 12. In some examples, a proximal portion of endovascular device 16 (e.g., second body portion 164 and third body portion 165, as described in connection with FIG. 3) remains outside of the vasculature of patient 12, and connects to medical device 14.

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

This disclosure includes the following non-limiting examples.

Example 1: A medical lead includes a first lead body portion including a first group of coiled wire filars; a second lead body portion including a second group of coiled wire filars; a third lead body portion including a third group of coiled wire filars; and a plurality of electrodes, each electrode of the plurality of electrodes electrically connected to at least one coiled wire filar of the first group of coiled wire filars; wherein the first group of coiled wire filars splits to form the second group of coiled wire filars and the third group of coiled wire filars, wherein the second group of coiled wire filars is configured to electrically connect to a first feedthrough of a medical device, and wherein the third group of coiled wire filars is configured to electrically connect to a second feedthrough of the medical device.

Example 2: The medical lead of example 1, wherein the second group of coiled wire filars and the third group of coiled wire filars are nonconcentric.

Example 3: The medical lead of examples 1 or 2, wherein the first group of coiled wire filars comprises dual concentric coils including at least an inner coil and an outer coil.

Example 4: The medical lead of example 3, wherein each coil of the dual concentric coils includes at least six filars.

Example 5: The medical lead of example 3, wherein each coil of the dual concentric coils includes at least eight filars.

Example 6: The medical lead of any of examples 3 through 5, wherein the dual concentric coils are wound in different directions.

Example 7: The medical lead of any of examples 3 through 5, wherein the dual concentric coils are wound in a common direction.

Example 8: The medical lead of examples 1 or 2, wherein the first group of coiled wire filars comprises a single coil.

Example 9: The medical lead of example 8, wherein the single coil includes at least twelve filars.

Example 10: The medical lead of example 8, wherein the single coil includes at least sixteen filars.

Example 11: The medical lead of any of examples 1 through 10, further includes a connector configured to electrically connect the first group of coiled wire filars to the second group of coiled wire filars and the third group of coiled wire filars.

Example 12: The medical lead of example 11, wherein the connector includes at least a first connector element and a second connector element, wherein the first connector element and the second connector element are configured to electrically connect at least some wire filars of the first group of coiled wire filars to wire filars of the second group of wire filars and the third group of wire filars.

Example 13: The medical lead of example 12, wherein the first connector element and the second connector element include a plurality of individual filar contact portions.

Example 14: The medical lead of any of examples 1 through 13, wherein one or more of the first group of coiled wire filars, the second group of coiled wire filars, and the third group of coiled wire filars includes a beta-titanium alloy.

Example 15: The medical lead of example 14, wherein the beta-titanium alloy comprises a Ti-15Mo alloy.

Example 16: The medical lead of any of examples 1 through 15, further includes an expandable structure at a distal portion of a lead body of the medical lead, the expandable structure configured to transform from a relatively low-profile delivery configuration to a deployed configuration in a blood vessel of a patient, wherein the plurality of electrodes are disposed on the expandable structure.

Example 17: A method includes introducing a medical lead into vasculature of a patient, the medical lead includes a first lead body portion including a first group of coiled wire filars, a second lead body portion including a second group of coiled wire filars, a third lead body portion including a third group of coiled wire filars, and a plurality of electrodes, each electrode of the plurality of electrodes electrically connected to at least one coiled wire filar of the first group of coiled wire filars, wherein the first group of coiled wire filars splits to form the second group of coiled wire filars and the third group of coiled wire filars, wherein the second group of coiled wire filars is configured to electrically connect to a first feedthrough of a medical device, and wherein the third group of coiled wire filars is configured to electrically connect to a second feedthrough of the medical device; and advancing the medical lead until the plurality of electrodes are adjacent a target location in the vasculature of the patient.

Example 18: The method of example 17, wherein the second group of coiled wire filars and the third group of coiled wire filars are nonconcentric.

Example 19: The method of examples 17 or 18, wherein the first group of coiled wire filars comprises dual concentric coils including at least an inner coil and an outer coil.

Example 20: The method of example 19, wherein each coil of the dual concentric coils includes at least six filars.

Example 21: The method of example 19, wherein each coil of the dual concentric coils includes at least eight filars.

Example 22: The method of any of examples 19 through 21, wherein the dual concentric coils are wound in different directions.

Example 23: The method of any of examples 19 through 21, wherein the dual concentric coils are wound in a common direction.

Example 24: The method of examples 17 or 18, wherein the first group of coiled wire filars comprises a single coil.

Example 25: The method of example 24, wherein the single coil includes at least twelve filars.

Example 26: The method of example 24, wherein the single coil includes at least sixteen filars.

Example 27: The method of any of examples 17 through 26, wherein the medical lead includes a connector configured to electrically connect the first group of coiled wire filars to the second group of coiled wire filars and the third group of coiled wire filars.

Example 28: The method of example 27, wherein the connector includes at least a first connector element and a second connector element, wherein the first connector element and the second connector element are configured to electrically connect at least some wire filars of the first group of coiled wire filars to wire filars of the second group of wire filars and the third group of wire filars.

Example 29: The method of example 28, wherein the first connector element and the second connector element include a plurality of individual filar contact portions.

Example 30: The method of any of examples 17 through 29, wherein one or more of the first group of coiled wire filars, the second group of coiled wire filars, and the third group of coiled wire filars includes a beta-titanium alloy.

Example 31: The method of example 30, wherein the beta-titanium alloy comprises a Ti-15Mo alloy.

Example 32: The method of any of examples 17 through 31, wherein the medical lead comprises an expandable structure at a distal portion of a lead body of the medical lead, the expandable structure configured to transform from a relatively low-profile delivery configuration to a deployed configuration in a blood vessel of a patient, wherein the plurality of electrodes are disposed on the expandable structure.

Example 33: A medical device system includes a medical lead including a lead body, the medical lead includes a first lead body portion including a first group of coiled wire filars, a second lead body portion including a second group of coiled wire filars, a third lead body portion including a third group of coiled wire filars, a first group of electrical contacts at a proximal portion of the second lead body portion, each electrical contact of the first group of electrical contacts electrically connected to respective filars of the second group of coiled wire filars, a second group of electrical contacts at a proximal portion of the third lead body portion, each electrical contact of the second group of electrical contacts electrically connected to respective filars of the third group of coiled wire filars, a plurality of electrodes, each electrode of the plurality of electrodes electrically connected to at least one coiled wire filar of the first group of coiled wire filars, and an expandable structure at a distal portion of the lead body configured to transform from a relatively low-profile delivery configuration to a deployed configuration in a blood vessel of a patient, wherein the plurality of electrodes are disposed on the expandable structure, wherein the first group of coiled wire filars splits to form the second group of coiled wire filars and the third group of coiled wire filars, wherein the second group of coiled wire filars is configured to electrically connect to a first feedthrough of a medical device via the first group of electrical contacts, and wherein the third group of coiled wire filars is configured to electrically connect to a second feedthrough of the medical device via the second group of electrical contacts; and a medical device configured to: control therapy delivery via the plurality of electrodes or sense a patient parameter via the plurality of electrodes.

Various examples of the disclosure have been described. Any combination of the described systems, operations, or functions is contemplated. These and other examples are within the scope of the following claims.

What is claimed is:

1. A medical lead comprising:
a first lead body portion including a first group of coiled wire filars;
a second lead body portion including a second group of coiled wire filars;
a third lead body portion including a third group of coiled wire filars; and
a plurality of electrodes, each electrode of the plurality of electrodes electrically connected to at least one coiled wire filar of the first group of coiled wire filars;
wherein the first group of coiled wire filars splits to form the second group of coiled wire filars and the third group of coiled wire filars,
wherein the second group of coiled wire filars is configured to electrically connect to a first feedthrough of a medical device, and
wherein the third group of coiled wire filars is configured to electrically connect to a second feedthrough of the medical device.

2. The medical lead of claim 1, wherein the second group of coiled wire filars and the third group of coiled wire filars are nonconcentric.

3. The medical lead of claim 1, wherein the first group of coiled wire filars comprises dual concentric coils including at least an inner coil and an outer coil.

4. The medical lead of claim 3, wherein each coil of the dual concentric coils includes at least six filars.

5. The medical lead of claim 3, wherein the dual concentric coils are wound in different directions.

6. The medical lead of claim 3, wherein the dual concentric coils are wound in a common direction.

7. The medical lead of claim 1, wherein the first group of coiled wire filars comprises a single coil.

8. The medical lead of claim 7, wherein the single coil includes at least twelve filars.

9. The medical lead of claim 1, further comprising:
a connector configured to electrically connect the first group of coiled wire filars to the second group of coiled wire filars and the third group of coiled wire filars.

10. The medical lead of claim 9, wherein the connector includes at least a first connector element and a second connector element, wherein the first connector element and the second connector element are configured to electrically connect at least some wire filars of the first group of coiled wire filars to wire filars of the second group of wire filars and the third group of wire filars.

11. The medical lead of claim 10, wherein the first connector element and the second connector element include a plurality of individual filar contact portions.

12. The medical lead of claim 1, further comprising:
an expandable structure at a distal portion of a lead body of the medical lead, the expandable structure configured to transform from a relatively low-profile delivery configuration to a deployed configuration in a blood vessel of a patient, wherein the plurality of electrodes are disposed on the expandable structure.

13. A method comprising:
introducing a medical lead into vasculature of a patient, the medical lead comprising:
a first lead body portion including a first group of coiled wire filars,
a second lead body portion including a second group of coiled wire filars,
a third lead body portion including a third group of coiled wire filars, and
a plurality of electrodes, each electrode of the plurality of electrodes electrically connected to at least one coiled wire filar of the first group of coiled wire filars,
wherein the first group of coiled wire filars splits to form the second group of coiled wire filars and the third group of coiled wire filars,
wherein the second group of coiled wire filars is configured to electrically connect to a first feedthrough of a medical device, and
wherein the third group of coiled wire filars is configured to electrically connect to a second feedthrough of the medical device; and
advancing the medical lead until the plurality of electrodes are adjacent a target location in the vasculature of the patient.

14. The method of claim 13, wherein the second group of coiled wire filars and the third group of coiled wire filars are nonconcentric.

15. The method of claim 13, wherein the first group of coiled wire filars comprises dual concentric coils including at least an inner coil and an outer coil.

16. The method of claim 13, wherein the first group of coiled wire filars comprises a single coil.

17. The method of claim 13, wherein the medical lead includes a connector configured to electrically connect the first group of coiled wire filars to the second group of coiled wire filars and the third group of coiled wire filars.

18. The method of claim 17, wherein the connector includes at least a first connector element and a second connector element, wherein the first connector element and the second connector element are configured to electrically connect at least some wire filars of the first group of coiled wire filars to wire filars of the second group of wire filars and the third group of wire filars.

19. The method of claim 18, wherein the first connector element and the second connector element include a plurality of individual filar contact portions.

20. A medical device system comprising:
a medical lead including a lead body, the medical lead comprising:
a first lead body portion including a first group of coiled wire filars,
a second lead body portion including a second group of coiled wire filars,
a third lead body portion including a third group of coiled wire filars,
a first group of electrical contacts at a proximal portion of the second lead body portion, each electrical contact of the first group of electrical contacts electrically connected to respective filars of the second group of coiled wire filars,
a second group of electrical contacts at a proximal portion of the third lead body portion, each electrical contact of the second group of electrical contacts electrically connected to respective filars of the third group of coiled wire filars,
a plurality of electrodes, each electrode of the plurality of electrodes electrically connected to at least one coiled wire filar of the first group of coiled wire filars, and
an expandable structure at a distal portion of the lead body configured to transform from a relatively low-profile delivery configuration to a deployed configuration in a blood vessel of a patient,
wherein the plurality of electrodes are disposed on the expandable structure,
wherein the first group of coiled wire filars splits to form the second group of coiled wire filars and the third group of coiled wire filars,
wherein the second group of coiled wire filars is configured to electrically connect to a first feedthrough of a medical device via the first group of electrical contacts, and
wherein the third group of coiled wire filars is configured to electrically connect to a second feedthrough of the medical device via the second group of electrical contacts; and
a medical device configured to:
control therapy delivery via the plurality of electrodes or sense a patient parameter via the plurality of electrodes.
